(19) United States
(12) Patent Application Publication
(10) Pub. No.: US 2016/0082211 A1
(43) Pub. Date: Mar. 24, 2016

(54) Adaptable Viewing Port for Endotracheal Tube

(71) Applicant: THE ADMINISTRATORS OF THE TULANE EDUCATIONAL FUND, New Orleans, LA (US)

(72) Inventors: Nicholas CHEDID, New Orleans, LA (US); Christopher COVER, New Orleans, LA (US); Scott KLEINPETER, Baton Rouge, LA (US); Gabriela NUNEZ, Huixquilucan (MX); Seth VIGNES, New Orleans, LA (US); Jaime PALOMINO, New Orleans, LA (US); James R. KORNDORFFER, JR., New Orleans, LA (US); Yashvir SANGWAN, Salisbury, MD (US)

(21) Appl. No.: 14/785,317

(22) PCT Filed: Apr. 17, 2014
(86) PCT No.: PCT/US14/34562

§ 371 Date: Oct. 16, 2015

Related U.S. Application Data
(60) Provisional application No. 61/813,352, filed on Apr. 18, 2013.

(51) Int. Cl. A61M 16/04; A61B 1/00; A61B 1/012; A61B 1/267

(52) U.S. Cl. CPC A61M 16/0402; A61M 16/0447; A61M 16/0493; A61B 1/2676; A61B 1/00082; A61B 1/012

(57) ABSTRACT

This disclosure provides a medical device adaptable to current endotracheal tubes having a first segment defining a first lumen with both a distal and proximal end, and a second segment defining a second lumen with both a distal and a proximal end. The first segment and the second segment may be attached to one another. The first segment's distal end extends farther than the second and is configured to receive a viewing device, such as a bronchoscope, whereas the second segment distal end is configured to receive a previously placed endotracheal tube. The device allows a single physician to perform tracheostomy procedures such as percutaneous procedures while reducing risks associated with such procedures.

### CROSS REFERENCE TO RELATED APPLICATIONS

This application claims priority to U.S. Provisional Patent Application No. 61/813,352 filed on Apr. 18, 2013 and entitled “Adaptable Viewing Port For Endotracheal Tube.” The entire contents of the above-identified application are hereby fully incorporated herein by reference.

### TECHNICAL FIELD

The disclosure relates to a medical device used for procedures that require an interior view of the larynx, trachea, and lungs. More specifically, this specification describes a medical device for use in the aid of procedures such as percutaneous tracheostomy and evaluation of laryngeal edema on an intubated patient.

### BACKGROUND

An alternative to the surgical tracheostomy, percutaneous dilatational tracheostomy (PDT) was introduced in 1985 by Ciaglia using the Seldinger technique. Since then, PDT has increased in popularity due to fewer complications and ability to be performed bedside in the ICU. Using a series of gauged needles and graded instruments, a stoma is created between the 2^{nd} or 3^{rd} tracheal ring in order for a patient to breathe without the use of an endotracheal tube (ET tube). The current procedure requires a pulmonologist to operate a bronchoscope and a Surgeon to perform the procedure. The Pulmonologist will insert the bronchoscope within the patient's ET tube and progresses the tip of the bronchoscope out of the end of the ET tube. The Surgeon then manually palpates the area of the future incision, while the pulmonologist slowly retracts the ET tube and the bronchoscope until the palpitations of the surgeon are visual. The surgeon then performs the PDT procedure while the pulmonologist maintains the internal visual.

The current procedure using the current equipment requires two highly trained individuals. The surgeon must guess the correct position at which to perform the incision and the Pulmonologist must predict the correct positioning of the bronchoscope in relation to the sensitive vocal cords as well as the incision site. Wrong guesses may result in damage to equipment or the loss of the airway.

Complications that arise during current PDT procedure are related to operational error caused by absence of visual aid during surgery. Unintended tracheal damage, tracheo-esophageal fistulas, pneumothorax, pnuemomediastinum, and uncontrolled bleeding are all major incidents, potentially fatal, that can occur due to misplacement of the gauged needle. Insertion of the initial gauged needle also may lead to equipment failure as in the case of puncture of the ET tube cuff or damage by the needle to the unprotected bronchoscope tip. During the current procedure the surgeon must retract the ET tube distally to the larynx. This increases the possibility of the ET tube becoming dislodged from the trachea if the tube is retracted too far. Complications arise from introducing the patient into a hypoxic environment while no ventilations are being administered. Other complications arise from obstruction of the ET-tube, via inserted brochonscope, which introduces a hypoxic and hypercarbic environment. To overcome hypoxic environments, ventilators can create a positive pressure environment inducing Auto-PEEP or pneumatic barotrauma.

Laryngeal Edema is a common pathological response to the presence of an endotracheal tube after any prolonged intubation. The continuous contact between the endotracheal tube and larynx results in edema of the vocal cords and surrounding tissue. This poses complications during the evaluation for extubation of the patients endotracheal tube. A current, popular methods performed by intensivist is called the Cuff-Leak Test. Initially developed for children with croup, it was adopted to evaluate the patient for the possibility of post-extubation stridor. The test is a simple and non-visual, the endotracheal tube cuff is deflated and the air-leak is measured with the relationship between air loss and degree of laryngeal edema being inversely related. This test is prone to false negatives due to the variability to the discrimination power. A wrong guess may result in the closure of an airway the inability for the patient to breath.

Therefore, there is a need for a medical device which can enable and maintain bronchoscopic visuals and supply continuous ventilations while eliminating or reducing complications inherent to the these types of procedures.

While certain novel features of this invention shown and described below are pointed out in the claims, the invention is not intended to be limited to the details specified, since a person of ordinary skill in the relevant art will understand that various omissions, modifications, substitutions and changes in the forms and details of the invention illustrated and in its operation may be made without departing in any way from the spirit of the present invention. No feature of the invention is critical or essential unless it is expressly stated as being “critical” or “essential.”

### SUMMARY

The present disclosure provides a medical device for use with existing endotracheal tubes comprising a first segment defining a first lumen and second segment defining a second lumen. The first and second segments comprise a proximal end and a distal end respectively. In certain example embodiments, the first segment is a longer length than the second segment and is adjacent to, and runs along a parallel axis with, the second segment. The first segment is sized to fit a medical device, such as a bronchoscope, and the second segment is sized to receive an in situ endotracheal tube. The first segment and the second segment may be derived from a single tubular component, or may be formed from separate tubular components joined circumferentially along a common axis. The distal ends of the first and second segments may terminate at an inflatable balloon. In certain example embodiments, the inflatable balloon is a laryngeal mask airway balloon.

In certain example embodiments, the first segment has a length of about 9 cm to about 24 cm. In certain example embodiments, the first segment has a Shore A durometer rating of about 50 to 80. In certain example embodiments, the first segment has an interior diameter of approximately 3.5 to 7 mm.

In certain example embodiments, the second segment has a length of about 4 cm to about 8 cm. In certain example embodiments, the second segment has a Shore A durometer rating of about 50 to 80. In certain example embodiments, the second segment has an inner diameter of about 7.5 mm to about 12 mm.

A cap may be placed on the proximal ends of the first segment, the second segment or both. The cap may further comprise a slit or circular opening in a top surface of the cap to receive a medical device in the case of the first segment, or an endotracheal tube in the case of the second segment. The slit or opening is configured to firmly grip the inserted medical device or endotracheal tube and help minimize potential air loss and prevent rotational and longitudinal motion. The cap may be made from silicone rubber, poly-vinyl chloride, or a similar polymer.

The device may further comprise a handle positioned between the first and second segment. The handle may be made of a hard plastic or a metal material and coated with a soft plastic covering. The handle may further comprise a cylindrical tube that acts as a bite blocker when the device is properly positioned within an airway. In certain example embodiments, the handle portion may be formed by a proximal end of the first segment, the handle portion having a larger outer diameter and higher durometer rating than the remainder of the first segment.

This disclosure provides a device that enables a single physician to perform intubation, diagnosis of laryngeal edemas, and tracheostomy procedures, such as percutaneous dilatational tracheostomy, while reducing risks associated with these types of procedures. The device may be used where an endotracheal tube has already been placed in a patient prior to the procedure. The device may also eliminate or reduce loss of airway, prevent the development of low oxygen and/or high carbon environments, and may eliminate the need for high pressure environments for such procedures.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

FIGS. 1A and 1B are diagrams of a double lumen medical device, in accordance with certain example embodiments.

FIG. 2 is a diagram showing a distal end cross-view of the double lumen medical device, in accordance with certain example embodiments.

FIG. 3 is a diagram showing a cross-sectional view of a first segment and second segment of the device, in accordance with certain example embodiments.

FIG. 4 is a diagram of a cap placed on the proximal ends of the first segment, second segment, or both, in accordance with certain example embodiments.

FIG. 5 is a diagram of a double lumen medical device further comprising a handle, in accordance with certain example embodiments.

### DETAILED DESCRIPTION OF THE EXAMPLE EMBODIMENTS

### Overview

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

Wherever the phrase “for example,” “such as,” “including” and the like are used herein, the phrase “and without limitation” is understood to follow unless explicitly stated otherwise. Similarly “an example,” “exemplary” and the like are understood to be non-limiting.

The terms “comprising” and “including” and “having” and “involving” (and similarly “comprises”, “includes,” “has,” and “involves”) and the like are used interchangeably and have the same meaning Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c.

Where ever the terms “a” or “an” are used, “one or more” is understood, unless such interpretation is nonsensical in context.

The terms “about” or “approximately” encompass the stated measured value and values within 1%, 2%, 3%, 4%, 5%, 6%, 7%, 8%, 9%, or 10% of the stated measured value.

This disclosure provides a medical device adaptable to current endotracheal tubes having a first segment defining a first lumen with both a distal and proximal end, and a second segment defining a second lumen with both a distal and a proximal end. The first segment and the second segment may be attached to one another. In certain example embodiments, the first segment's distal end may extend farther than the second segment and is configured to receive surgical equipment, such as but not limited to, a bronchoscope, whereas the second segment's distal end is configured to receive a previously placed endotracheal tube. In certain example embodiments, the first segment is configured to receive a pediatric bronchoscope. In certain example embodiments, the first segment is configured to receive an adult bronchoscope. In some embodiments, the first segment is thick enough, while maintaining its ability to receive surgical equipment, to act as a handle. In some embodiments, the first and second segments are distinct separate tubes attached circumferentially along a center line. In further embodiments, the first and second segments are two separate tubes within a single, extruded multi-lumen tubing. In further embodiments, the first and second segments are attached at their distal ends via an inflatable balloon. In further embodiments, the first and second segments are attached via a laryngeal mask airway balloon. In other example embodiments, the device may further comprise a hard plastic or metal handle with a soft plastic coating. In certain example embodiments, the handle extends from between the first and second segments. In some example embodiments, the handle extends posteriorly to the second segment.

Turning now to the drawings, in which like numerals represent like (but not necessarily identical) elements throughout the figures, example embodiments are described in detail.

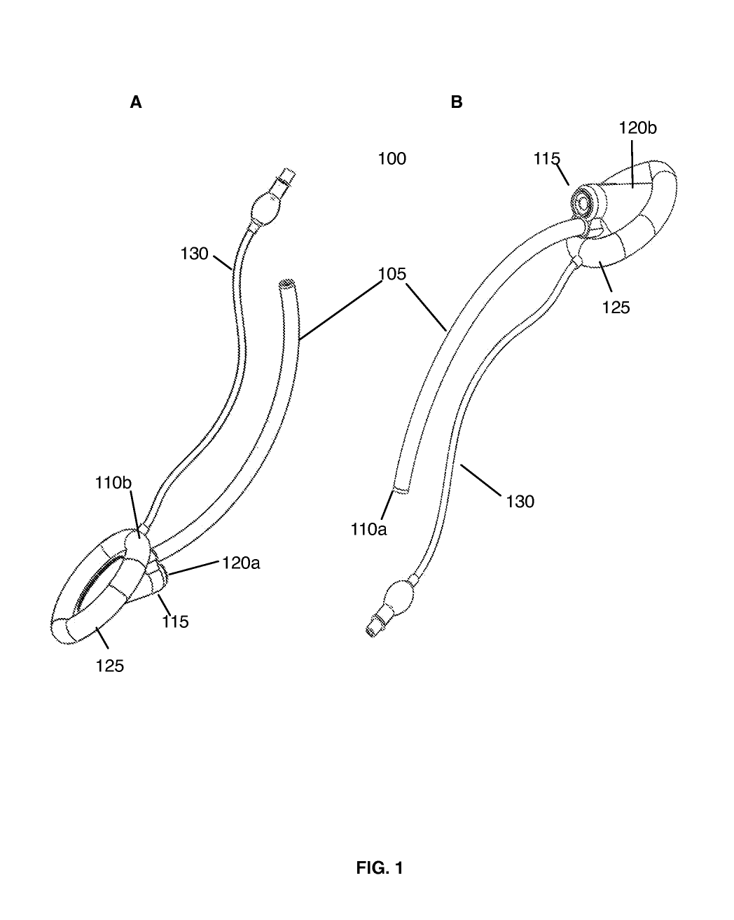

FIGS. 1A and B provide front and back isotpropic views of an example double lumen device 100. The device 100 comprises a first segment 105 and a second segment 115. The first segment 105, or viewing tube, receives surgical equipment, such as a bronchoscope. The second segment 115, or endotracheal tube or sheath, receives an existing in situ endotracheal tube previously placed in a subject patient's airway. The first segment 105 defines a first lumen and comprises a proximal end 110a, or operator end, and a distal end 110b. The second segment 115 likewise defines a second lumen and comprises a proximal end 120a and a distal end 120b. The distal ends 110b and 120b may terminate in an inflatable balloon 125. The device 100 may further comprise a balloon port 130 for inflating the balloon 125 once properly placed within a patient's airway. In certain example embodiments, the balloon may be a laryngeal mask airway balloon (LMA). In certain example embodiments, the volume of the balloon will be graded in respect to the inner diameter of the second segment 115. Grading with respect to inner diameter embodies a variation of inner diameter sizes due to variation of endotracheal tube outer diameter size. In some embodiments the size of the device will be graded in proportion to a patients weight. This is done in order to account for patient variability. In some embodiments, the distal tip of the inflatable balloon 125 will contain a separate lumen to allow a clear path for epigastric aspirated material (not shown).

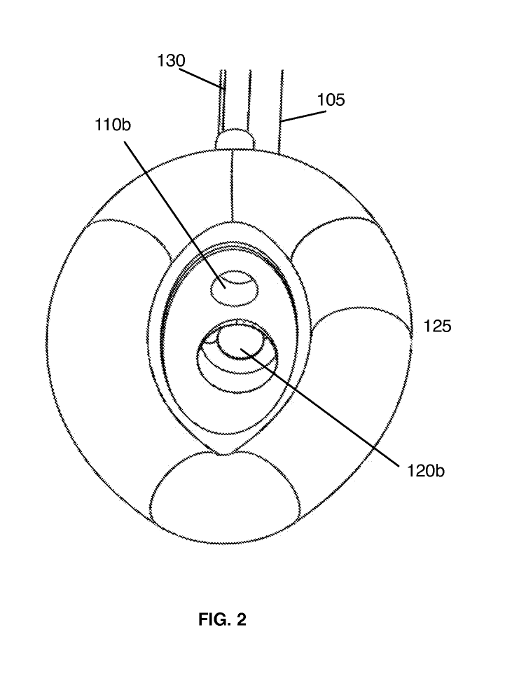

FIG. 2 shows a distal end cross-view of the device 100 and the distal openings 110b and 120b of the first segment 105 and second segment 115 within the balloon 125. In certain example embodiments, the first segment 105 may be positioned superior to the second segment. In certain other example embodiments, the first segment 105 may be positioned posterior to the second segment 115.

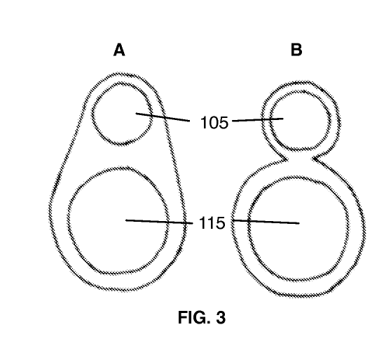

FIG. 3 shows a cross-sectional view of the first segment 105 and second segment 115. The first segment 105 and second segment 115 may be defined from a single tubular component. FIG. 3A. Alternatively, the first segment 105 and the second segment 115 may comprise two separate tubes joined circumferentially along a common axis. FIG. 3B.

The length of the first segment 105 may be sized so that the distal end 110b extends just distal to a patient's vocal cords upon proper placement of the balloon 125. In certain example embodiments, the length of the first segment 105 is between 9 cm and 21 cm. In certain other example embodiments, the length of the first segment 105 is between about 9 cm, about 10 cm, about 11 cm, about 12 cm, about 13 cm, about 15 cm, about 16 cm, about 17 cm, about 18 cm, about 19 cm, about 20 cm, or about 21 cm. The viewing tube 105 may be marked on an exterior surface with a depth gauge to determine the depth of insertion. In certain example embodiments the diameter of the viewing tube is between 3.5 and 7 mm. In certain example embodiments the first segment 105 is a sufficient size to receive a pediatric bronchoscope. In certain other example embodiments, the first segment 105 is a sufficient size to receive an adult bronchoscope. In certain example embodiments, the first segment may be wide enough to carry an esophageal drainage tube in addition to the medical device.

The size and length of the second segment 115 may be sized according to endotracheal tube sizing. In certain example embodiment, the length of the second segment 115 may depend on an inner diameter of the second segment 115. In certain example embodiments, the second segment 115 has an inner diameter of about 8 mm to about 12 mm, and a length of about 6 cm to about 8 cm. In another example embodiment, the second segment 115 has an inner diameter of about 12 mm and a length of about 8 cm. In another example embodiment, the second segment 115 has an inner diameter of about 10 mm and a length of about 7 cm. In other example embodiments, the second segment 115 has an inner diameter of about 8 cm and a length of about 6 cm.

The sizing of the second segment, the sizing of the first segment, and the distances between the distal ends of the tubes may vary from the specifically exemplified embodiments, such as that it ranges in length from about 10 cm to about 14 cm and in diameter from about 18 mm to about 25 mm, can determine appropriate functional size.

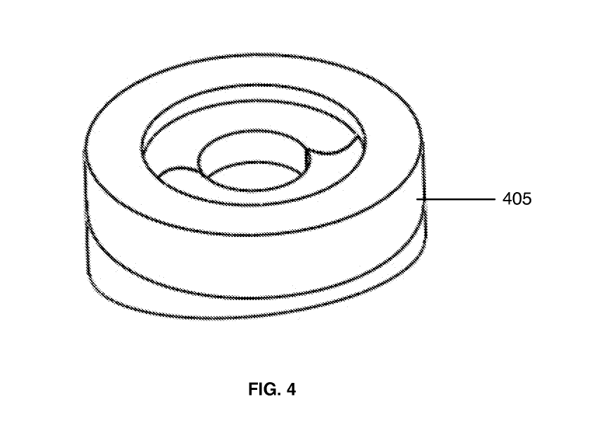

In certain example embodiments, a cap is placed on the proximal end of the first segment, the second segment or both. FIG. 4 provides a diagram of an example cap 405. The cap 405 allows the device 100 to receive endotracheal tubes or medical equipment of differing diameters without significant air loss or rotational or longitudinal movement. In certain example embodiments, cape 405 has a slit or circular opening at a top end of the cap 405 that will deform to hug the walls of a received endotracheal tube or piece of surgical equipment. In certain example embodiments, the cap 405 is made of silicone rubber, poly-vinyl chloride (PVC), or similar material. In certain example embodiments, the cap is a medical grade silicone rubber cap. The inner diameter of the cap 405 coincides with the outer diameter of the corresponding segment on which it is placed. In addition the length of a cap may be proportional to the length of the segment over which it is placed.

In certain example embodiments, the durometer rating or stiffness of the first segment 105 may be increased to increase structural support while using the device 100. In some embodiments, the Shore A durometer rating or stiffness of the second segment 115 may be lowered to ease implementation and promote contouring of the device within the oropharynx.

In certain example embodiments the distal tip 110b of the first segment 105 may be beveled to facilitate passing through the vocal chords. In certain example embodiments, the first segment 105 may have a slight curvature to its length. In other embodiments, the first segment 105 may be straight but bendable. In certain example embodiments, the first segment 105 is flush with the backplate of the device 100 or back surface of the balloon 125.

In certain example embodiments, the first segment 105 may be separable from the main device 100. In such embodiments, the first segment 105 will be adjustable along a proximal/distal scale through a series of removable segments similar to the caps used on the proximal tips of the first segment 105 and second segment 115. For example, the length of the first segment 105 may be increased or decreased by a corresponding addition or removal of segments. In other example embodiments, the second segment 115 may further include tracks with corresponding locking mechanisms on the viewing tube 105 to facilitate circumferential locking and longitudinal, parallel movement of the viewing tube 105. Alternatively, the first segment may comprise a track that can be extended to receive additional segments. The additional segments are slid over the track and locked in place by a locking mechanism.

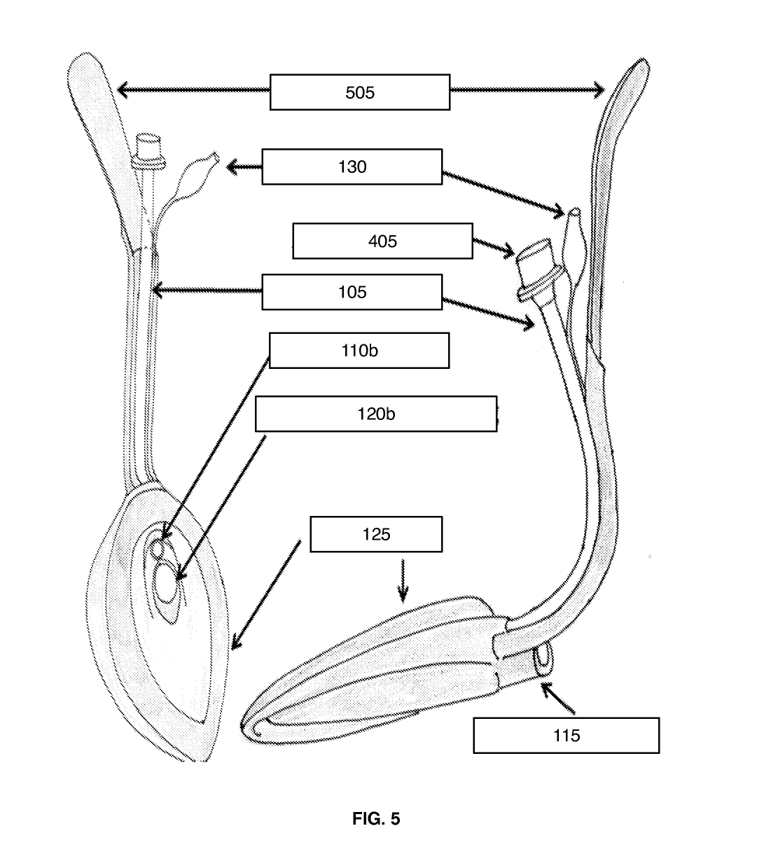

FIG. 5 provides a diagram of an alternative embodiment of the device 100 further comprising a handle 505. The handle 505 may be positioned between the first segment 105 and the second segment 115. In certain example embodiments, the handle 105 may comprise a groove or channel in which houses a portion of the first segment 105. The handle 505 may be made from metal or a hard biocompatible plastic. The handle 505 is covered by a layer of soft plastic. The soft plastic prevents local tissue damage during use of the device 100. The handle may allow an operator to intubate the device with minimal complication, effort, and time by improving operator control of the device 100. In certain example embodiments, the handle will house a cylindrical tube at the location of a patient's teeth so that, after intubation, the cylinder will act as a bite blocker to protect the bronchoscope or chosen medical device. In certain example embodiments, the handle will house a locking device to lock the first segment 105 in place if the first segment 105 is separable and/or adjustable. The length of the handle will protrude from between the patients teeth towards the operator. In certain example embodiments, the handle 505 will turn 90° towards the top of the patient's head. In certain other example embodiments, the handle 505 will turn 90° towards the patient's feet. In certain example embodiments, the handle 505 will be located dorsal to the first segment 105. In certain example embodiments, the handle 505 will be part of the first segment 105, the handle portion having a larger outer diameter and higher durometer rating than the rest of the first segment 105 while maintaining the same inner diameter. In said example embodiments, the handle portion of the first segment 105 will be cylindrical and may contain finger grooves to aid in insertion. In certain example embodiments, the first segment may be wide enough to carry an esophageal drainage tube in addition to the medical device.

In operation, the device 105 is inserted into a patient with proper endotracheal tube intubation wherein the first segment is position anteriorly and the second segment is slid over an existing endotracheal tube posteriorly. Prior to placement, the in situ endotracheal tube balloon is deflated and the air port is passed through the endotracheal sheath. The ventilator is then disconnected, the adaptor removed, and the second segment 115 is placed over the existing endotracheal tube. The adaptor is then reconnected to the endotracheal tube, the ventilator reconnected, and the balloon of the endotracheal tube re-inflated. The device 100 is then guided down the length of the endotracheal tube with the viewing tube 105 being used as the stiff ‘backbone’ to correctly place the device within the oropharynx. In some embodiments, markings on the tubular component defining the first segment will enable an operator to confirm the placement of the sheath and/or balloon.

In certain example embodiments, the device 100 is placed using a laryngoscope for correct placement. In some embodiments, the bronchoscope will be placed within the first segment 105 before being placed and used to correctly place the device 100 in the oropharynx with the distal end of the first segment 105 being positioned slightly past the vocal cords. Alternatively, the bronchoscope may be inserted after the device 100 is placed within the oropharynx and adjusted so that the distal tip of the first segment 105 is just beyond the vocal cords.

The first segment 105 is sized to snugly fit the bronchoscope. In certain example embodiments, the presence of a cap 405 at the proximal tip of the first segment 105 prevents rotational and longitudinal movement. After an ideal view has been established, the bronchoscope can then be placed on a table or hung on a hook and the procedure continued. Adjustments can be made during the procedure by a respiratory therapist or other trained operator.

Once correctly placed, the balloon 125 of the device 100 is inflated and the balloon of the endotracheal tube deflated. As a physician palpates the area of the incision, the endotracheal tube is withdrawn or advanced slowly until the distal tip of the endotracheal tube is clear of the incision site. This view is acquired by virtue of the bronchoscope inserted via the first segment 105.

In the case of diagnosing laryngeal edema, the device 100 is properly placed as described above and a standard Cuff Leak Test is performed by deflating the endotracheal tube cuff. In contrast to the standard Cuff Leak Test, the placement of the device allows for a visual while the endotracheal tube cuff is deflated and air leak measured. Accordingly, false negatives due to variability in the discrimination of the operator are reduced.

In some embodiments, procedures using the medical device disclosed herein may reduce risks of complications associated with conventional procedures using conventional equipment. Use of the device as described may eliminate or reduce loss of airway, prevent low oxygen/high carbon environments, and may eliminate the need for high pressure environments.

1. A device for use with existing endotracheal tubes comprising, a first segment defining a first lumen and comprising a proximal and a distal end, and a second segment defining a second lumen and comprising a proximal and distal end, wherein the first segment is adjacent to, and runs along a parallel axis with, the second segment, and wherein the first segment is sized to accommodate a bronchoscope and the second segment is sized to accommodate an endotracheal tube.

2. The device of claim 1, wherein the first segment and the second segment are defined within a single tubular component.

3. The device of claim 1, wherein the first segment and the second segment are defined by separate tubular components that are joined circumferentially along a common axis.

4. The device of claim 1, wherein the distal ends of the first segment and the second segment attach to an inflatable balloon.

5. The device of claim 4, wherein the balloon is a laryngeal mask airway balloon.

6. The device of claim 1, wherein the first segment, the second segment, or both, further comprise a cap at the proximal end, the cap comprising a slit in a top surface of the cap.

7. The device of claim 6, wherein the cap is made of silicon rubber or poly-vinyl chloride.

8. The device of claim 1, wherein the distal tip of the first segment may be beveled.

9. The device of claim 1, wherein the first segment has a Shore A durometer rating of between approximately 50 and 80, and the second segment has a durometer reading of between approximately 50 and 80.

10. The device of claim 1, wherein the first segment comprises one or more removable or slidable segments to facilitate adjustments to the length of the first segment.

11. The device of claim 1, wherein the second segment comprises a set of tracks and the first segment comprises a set of corresponding locking mechanisms to facilitate circumferential locking of the first and second segment.

12. The device of claim 1, further comprising a handle, the handle being positioned between the first and second segment and made of a hard plastic or metal material, wherein the hard plastic or metal material is covered with a soft plastic layer.

13. The device of claim 12, wherein the handle comprises a groove or channel that houses a portion of the first segment.

14. The device of claim 12, wherein the handle further comprises bite blocker comprising a cylindrical tube located on the handle so as to be positioned near a subject's teeth after intubation of the device.

15. The device of claim 1, wherein the first segment comprises a handle portion on the distal end, the handle portion having a larger outer diameter and higher durometer rating than the rest of the first segment and a same inner diameter as the rest of the first segment.

16. The device of claim 15, wherein the first segment further comprises an esophageal drainage tube.

17. The device of claim 1, wherein the length of the first segment is between 9 cm and 24 cm, and the second segment has a length of between 4 cm and 8 cm.

18. A method of performing a tracheostomy procedure comprising:
inserting the device of claim 1 into the oropharynx of a subject with an existing endotracheal tube, wherein the second segment is inserted over the existing endotracheal tube, and the first segment is placed slightly distal to vocal chords of the subject;
inflating the inflatable balloon of the device;
deflating the balloon of the existing endotracheal device;
inserting a bronchoscope into the first segment; and
removing the existing endotracheal device until the distal tip of the existing endotracheal device is just clear of a target incision cite as shown by images captured using the bronchoscope.

19. The method of claim 18 wherein the first segment and the second segment of the device are defined within a single tubular component.

20. The method of claim 18 wherein the first segment and the second segment of the device are defined by separate tubular components that are joined circumferentially along a common axis.
